(19) United States
(12) Patent Application Publication
(10) Pub. No.: US 2004/0086674 A1
(43) Pub. Date: May 6, 2004

(54) Laser sintering process and devices made therefrom

(76) Inventor: Thomas J. Holman, Minneapolis, MN (US)

Correspondence Address: VIDAS, ARRETT & STEINKRAUS, P.A., 6109 BLUE CIRCLE DRIVE, SUITE 2000, MINNETONKA, MN, 55343-9185, US

(21) Appl. No.: 10/286,281

(22) Filed: Nov. 1, 2002

(51) Int. Cl. B32B 1/08; A61L 2/00; B05D 3/00

(52) U.S. Cl. 428/36.9; 427/2.1; 427/554

(57) ABSTRACT

A medical device and method of manufacturing same comprises laser sintering a pattern of powdered metal and/or a dispersion of polymer material to an tubular member such as a catheter and/or catheter balloon.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

1. Field of the Invention

This invention relates to medical devices, their preparation and production. The invention is particularly directed to the field of intravascular medical devices, and more particularly to the field of catheters such as angioplasty, neurological and guide catheters, among others, which may be used in various medical procedures such as percutaneous transluminal angioplasty (PTA), percutaneous transluminal coronary angioplasty (PTCA) as well as in procedures involving the placement of medicines and medical devices within the body.

Some embodiments of the invention are directed to all forms of catheters which may be advanced through a body lumen or vessel. Some examples of catheters are over-the-wire (OTW) catheters, such as are described in U.S. Pat. No. 5,047,045; single-operator-exchange (SOE) balloon catheters, such as are described in U.S. Pat. No. 5,156,594 and U.S. Pat. No. 5,549,552. Other examples of catheters which may incorporate the unique features of the present invention may include rapid-exchange catheters, guide catheters, etc.

2. Description of the Related Art

Intravascular diseases are commonly treated by relatively non-invasive techniques such as PTA and PTCA. These angioplasty techniques typically involve the use of a balloon catheter. In these procedures, a balloon catheter is advanced through the vasculature of a patient such that the balloon is positioned proximate a restriction in a diseased vessel. The balloon is then inflated and the restriction in the vessel is opened. In other uses a catheter may be used to deliver an endoprosthesis such as a stent, graft, stent-graft, vena cava filter or other implantable device or devices herein after collectively referred to as a stent or stents. Where a stent is to be delivered into a body lumen the catheter may include one or more inflatable portions or balloons. Typically, the stent is retained in the predelivery state about the catheter shaft, or a portion thereof such as a balloon, by crimping and/or through the use of a retaining mechanism such as sleeve, sheath or sock.

Many procedures make use of a guide catheter positioned within the vascular system of a patient. The guide catheter assists in transporting a balloon dilation catheter, or other form of treatment catheter, to the portion of the vessel requiring treatment or inspection. The guide catheter is urged through the vasculature of the patient until its distal end is proximate the restriction. The balloon catheter may then be fed through a lumen in the guide catheter.

Guide catheters and other catheter types such as a dilatation or medical device delivery catheters must possess a level of rigidity which will allow it to traverse tortious pathways through blood vessels in a manner that minimizes trauma. The catheter must be capable of being advanced through the vascular system without folding or buckling despite application of longitudinal and/or rotational forces upon the catheter.

Balloons and balloon catheters may be particularly useful for the delivery of expandable, implantable medical devices such as stents, grafts, stent-grafts, vena cava filters, hereinafter referred to cumulatively as stents. Stents and catheters used in their delivery are commonly used and as such their structure and function are well known.

A stent is a generally cylindrical prosthesis introduced via a catheter into a lumen of a body vessel in a configuration having a generally reduced diameter and then expanded to the diameter of the vessel. In its expanded configuration, the stent supports and reinforces the vessel walls while maintaining the vessel in an open, unobstructed condition.

Self-expanding, inflation assisted expandable and inflation expandable stents are well known and widely available in a variety of designs and configurations. In using such stents and other expandable, implantable medical devices, it is necessary to position the expandable, implantable medical device in a precise location within a body lumen. This goal is rendered more difficult because slippage may occur during insertion of the expandable, implantable medical device through a guide catheter or during deployment of the expandable, implantable medical device. To facilitate the proper positioning of an expandable, implantable medical device, it is desirable to prevent any unwanted relative movement between any of the expandable, implantable medical device, the balloon, the catheter and the interior of the vessel.

The issue of slippage of an expandable, implantable medical device relative to a balloon has been dealt with in several different ways including by varying the coefficient of friction of the exposed portion of a balloon between the uninflated and inflated states of the balloon. Another approach involves providing a balloon with enlarged ends and a middle section of reduced diameter to retain a stent. Yet another approach involves encapsulating a stent with a balloon. Other approaches are non-balloon based, providing stent retention devices that extend from the catheter and engage the stent.

Patents, publications and applications of interest include U.S. Pat. No. 5,503,631 to Onishi, U.S. Pat. No. 5,545,132 to Fagan, U.S. Pat. No. 5,746,745 to Abele et al., U.S. Pat. No. 5,423,745 to Todd et al., U.S. Pat. No. 5,487,730 to Marcadis et al., U.S. Pat. No. 5,250,070 to Parodi, U.S. Pat. No. 4,927,412 to Menasche, U.S. Pat. No. 5,836,965 to Jendersee et al., U.S. Pat. No. 6,120,522 to Vrba et al., and WO 94/23787.

Another approach for providing improved securement of the stent to the balloon and/or catheter shaft includes providing the outer surface of the balloon and/or catheter shaft with surface features such as bumps, ridges, ribs, among others, to provide a surface to which the stent may be readily secured to prior to delivery.

Without limiting the scope of the invention, a brief summary of various embodiments of the invention is set forth below. Additional details of the summarized embodiments of the invention and/or additional embodiments of the invention may be found in the Detailed Description of the Invention below.

A brief abstract of the technical disclosure in the specification is provided as well for the purposes of complying with 37 C.F.R. 1.72.

The entire content of all of patents or other references listed within the resent patent application are incorporated herein by reference.

### BRIEF SUMMARY OF THE INVENTION

The present invention is directed to a variety of embodiments. At least one embodiment is directed to a catheter having a predetermined portion, such as at least a portion of a balloon, provided with external surface features, such as a texture. Preferably, the surface features are provided to the catheter by a unique laser sintering method such as is described in detail below.

The unique laser sintering method described below is utilized to provide the surface of a catheter, balloon or other device with a variety of surface features such as a roughened or textured surface. The textured surface may comprise any of a variety of patterns or designs of bumps, grooves, ribs, ridges, or any other desired surface feature.

In some embodiments, the portion (or portions) of the catheter having the surface features is provided with improved or desired physical characteristics such as: improved flexibility, improved stiffness, improved trackability, etc. Where the catheter is utilized with a stent or other implantable device, the surface features on the catheter can provide the stent with improved engagement characteristics. This improved engagement provides a medical device delivery system with improved stent deployment accuracy which reduces slippage of the stent during catheter advancement as well as during deployment of the stent to a desired body location

In accordance with at least one embodiment of the invention, a tubular device may be provided with desired characteristics, such as those mentioned above, by coating a surface of the tube with a polymer coated powdered metal and/or dispersion of polymer material. Such a coating may provide the tube with surface features such as mechanical locks, hubs, ribs, bumps, or other protrusions that may be useful for mechanically engaging a stent. Other surface features, such as rings and lines may provide portions of the tube, such as a balloon or other portions, with increased rigidity to improve trackability or alter inflation characteristics, etc.

In at least one embodiment of the invention a polymeric tubular member such as a catheter or balloon may be completely or selectively coated with a material coating of powdered metal and/or dispersion of polymer. In some embodiments the coating of material is applied by laser sintering the material onto the tubular member according to a predetermined pattern.

In some embodiments, sintered powdered metal on a tubular member may be used to join the tubular member to a second member by welding.

Additional details and/or embodiments of the invention are provided below.

### BRIEF DESCRIPTION OF THE SEVERAL VIEWS OF THE DRAWINGS

A detailed description of the invention is hereafter described with specific reference being made to the following drawings.

FIGS. 1-3 show a method for providing a tubular member with a pattern of laser sintered material, with FIG. 1 showing a perspective view of a tubular member having a coating of material suitable for laser sintering.

FIG. 2 is a perspective view of the tubular member of FIG. 1 wherein a design pattern is shown on the coating of material.

FIG. 3 is a perspective view of the tubular member of FIG. 2 shown following application of laser energy to the coating of material according to the design pattern to provide a pattern of sintered material.

FIG. 4 is a block diagram representation of a method of providing a tubular member with a pattern of laser sintered material.

FIG. 5 is a side view of a tubular member provided with a pattern of converging stripes of laser sintered material made according to the method shown in FIGS. 1-4.

FIG. 6 is a side view of a tubular member provided with a helical stripe of laser sintered material made according to the method shown in FIGS. 1-4.

FIG. 7 is a side view a tubular member provided with a pattern of parallel longitudinal stripes of laser sintered material made according to the method shown in FIGS. 1-4.

FIG. 8 is a side view of a catheter balloon provided with a pattern of angularly oriented stripes of laser sintered material made according to the method shown in FIGS. 1-4.

FIG. 9 is a side view of a catheter balloon provided with a plurality of longitudinally oriented stripes of laser sintered material made according to the method shown in FIGS. 1-4.

FIG. 10 is a side view of a catheter balloon provided with a pattern of laser sintered material made according to the method shown in FIGS. 1-4.

FIG. 11 is a side view of a catheter balloon provided with a pattern of laser sintered material made according to the method shown in FIGS. 1-4.

FIG. 12 is a perspective view of a stent on a delivery system wherein a stent retaining portion of a balloon catheter is provided with a pattern of laser sintered material suitable for engaging the stent.

FIG. 13 is a cross-sectional view of a tubular member having a raised pattern of laser sintered material on its outer surface.

FIG. 14 is a cross-sectional view of a tubular member having a pattern of peaks and valleys provided by the laser sintering method shown in FIGS. 1-4.

FIGS. 15-17 show a method for applying a textured surface to a tubular member with FIG. 15 showing is a perspective view of the tubular member having a coated band of material suitable for laser sintering.

FIG. 16 is a perspective view of the tubular member of FIG. 15 wherein a heat shrink has been applied to the tubular member over the band.

FIG. 17 is a perspective view of the tubular member of FIG. 16 shown following application of laser energy to the band and removal of the heat shrink, the tube being provided with a textured surface of laser sintered material.

FIGS. 18-20 show the steps of a method for welding two tubular member together by laser sintering a coating of material placed on the outside surface of the inner tube thereby bonding the inner tube and the outer tube together.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

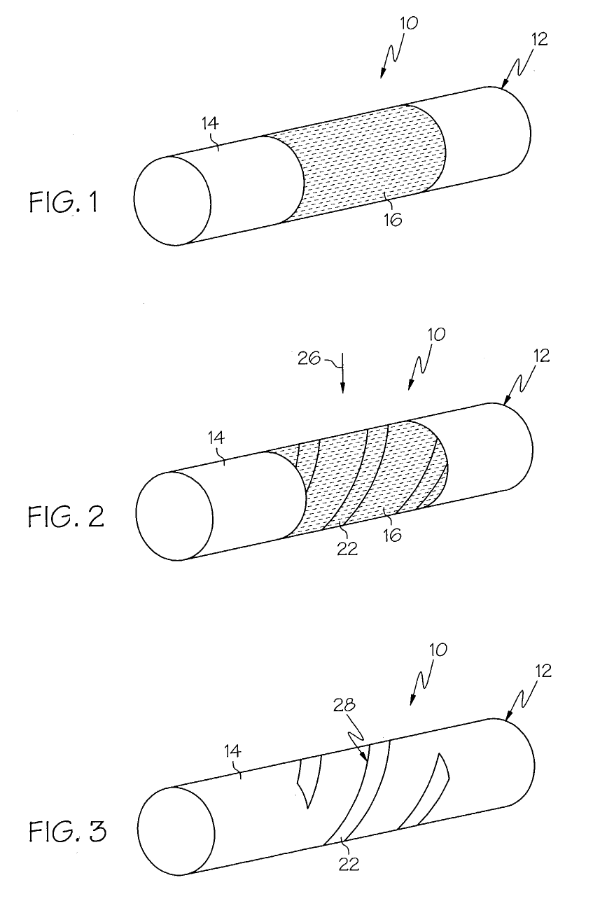

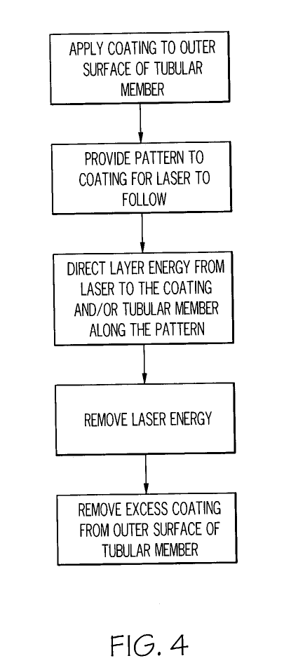

The present invention includes many different embodiments. a first embodiment of the invention is directed to a method of providing a tubular member with a predetermined pattern of laser sintered material such as is summarized in the block diagram of FIG. 4. The components of the method are illustrated in FIGS. 1-3. FIG. 1 shows a tubular member, shown generally at 10, having an outside surface 14. A predetermined portion of the outside surface 14 is coated with a coating 16. Coating 16 comprises a powdered non-polymeric substance such as metal and/or a polymer dispersion.

Coating 16 may be provided by known processes such as Precision Metal Injection Molding (PMIM) wherein various sizes of fine particles of metal or metal powder are coated with one or more polymers, such as for example a thermoset polymer and polyethylene glycol among others.

In the various embodiments shown and described herein, tubular member 10 may be any type of elongate tube, such as a tubular medical device suitable for insertion and advancement through a body lumen. Preferably, the tubular member 10 is a catheter 12 or portion thereof such as a balloon 20 shown in FIGS. 8-11.

Numerous types and configurations of such tubular medical devices are known and the term “catheter” as used herein is merely a convenient term used to designate all such devices.

In the various embodiments described herein, catheter 12, of FIGS. 1-3, or balloon 20, of FIGS. 8-12, may be manufactured from a variety of suitable materials. A typical catheter material may include but is not limited to a wide variety of polymeric substances such as: polyester/polyether elastomers such as Arnitel™ available from DSM Engineering; polyurethane-polyether polymers, such as Tecothane™ and/or Tecoplast™ both being available from Thermedics, Inc.; polyester-polyurethanes, such as Pellethane™ sold by Dow Chemical; polyester-polyurethanes, such as Estane™ sold by BF Goodrich; polyether block amides (PEBA), such as Pebax™ available from Elf Atochem; styrene-butadien-styrene triblock copolymers, such as Kraton™ sold by Shell Chemical company; styrenic block copolymers; polyurethanes; silicone rubber; natural rubber; copolyesters; polyamides; EPDM rubber/polyolefin; nitril rubber/PVC; fluoroelastomers; butyl rubber; epichlorohydrin; block copolymers; polyethylene terephthalate (PET); polyethylene naphthalate (PEN); polybutylene terephthalate (PBT); polytrimethylene terephthalate (PTT); fluoropolymers; polyolefins; polystyrene; polyvinyl chloride (PVC); acrylonitrile-butadiene-styrene polymers; polyacrylonitrile; polyacrylate; vinyl acetate polymer; cellulose plastics; polyacetal; polyethers; polycarbonates; polyphenylene sulfide; polyarylethersulfones; polyaryletherketones; polytetrafluoroethylene; polyamide copolymer, such as MXD6™ available from Mitsubishi Gas Chemical Co. or Cristamid™ available from Atofina, etc. Other materials are also know to those of skill in the art which may be suitable for use in constructing the present catheter, balloon, or portions thereof.

Returning to the method of preparing the tubular member 10 depicted in FIGS. 1-3, following the application of the coating 16 to the outside surface 14, such as is shown in FIG. 1, a design or pattern 22 is provided to the coating 16 as shown in FIG. 2. Pattern 22 may be provided by a stencil, mask or other device for providing a predetermined pattern to an object. Alternatively, the pattern 22 is a preprogrammed path that a laser or other energy transmission device, represented in the block diagram of FIG. 4, is provided with in order to direct laser energy to the coating 16 and tubular member 10 to laser sinter the coating 16 to the tubular member 10 according to the desired pattern 22. The laser may be any type of laser including but not limited to a diode laser, YAG laser, infrared (IR) laser, ultraviolet (UV) laser, etc.

Lasers are well known devices for transmitting energy, and a laser as provided for in the present invention is selected to provide the necessary frequency and/or wavelength necessary to at least partially melt at least one of the material of the tubular member 10 and the coating 16, when laser energy is transmitted thereto. Therefore, a laser as used according to the present method, may be any type of laser having a wavelength that is at least partially absorbable by the material of the coating 16 and/or the tubular member 10. Depending on the type of laser used, the particular energy absorbing properties of the materials and time energy is applied to the materials, the various components of the tubular member 10 may be selectively manipulated to various extents by selectively absorbing laser energy in accordance with their respective absorbative properties of the particular wavelength of laser energy applied thereto. For example, a particular type of YAG laser will transmit laser energy having a wavelength of about 1054 nanometers. A particular type of diode laser transmits laser light within a range of about 750 nanometers to about 950 nanometers, and a particular type of carbon dioxide laser will transmit light at about 10,600 nanometers.

One or more of the materials of the coatings 16 and/or tubular member 10 should be selected in order to at least partially absorb the wavelength of the laser energy to be transmitted thereto. For example if a diode laser is used and the tubular member is constructed of a material such as clear Pebax which is substantially transparent to the laser energy transmitted by the laser, then the coating 16 may include a nylon or other material that is colored to at least partially absorb the particular wavelength of energy transmitted by the diode laser.

As indicated by the steps shown in FIG. 4 and shown in FIG. 2, a laser directs laser energy, indicated by arrow 26, to the coating 16 along the pattern 22. The laser energy 26 may be transparent to the coating 16 and cause a momentary melting of the underlying tube material. This momentary melting, followed by resolidification of the effected tubular member 10 and/or the coating 16, will embed or incorporate the coating 16 of the pattern 22 into the tubular member 10 when the laser energy and the member 10 is allowed to cool. As a result, the effected portion of the tubular member 10 and coating 16 provides outer surface 14 of the tubular member with a pattern 22 of laser sintered material 28 such as is shown in FIG. 3. Alternatively, the coating 16 along the pattern 22 is at least partially melted, or both the coating 16 and the material of the tubular member 10 are at least partially melted to form the pattern 22 of laser sintered material 28.

Upon removal of the laser energy 26, the melted portion of the coating 16 and/or material of the tubular member 10 will resolidify to provide the completed tubular member 10 with the pattern 22 of laser sintered material 28 shown in FIG. 3. Once the sintered material 28 has been provided, the excess coating 16 that is outside of the pattern 22 is removed from the outer surface 14.

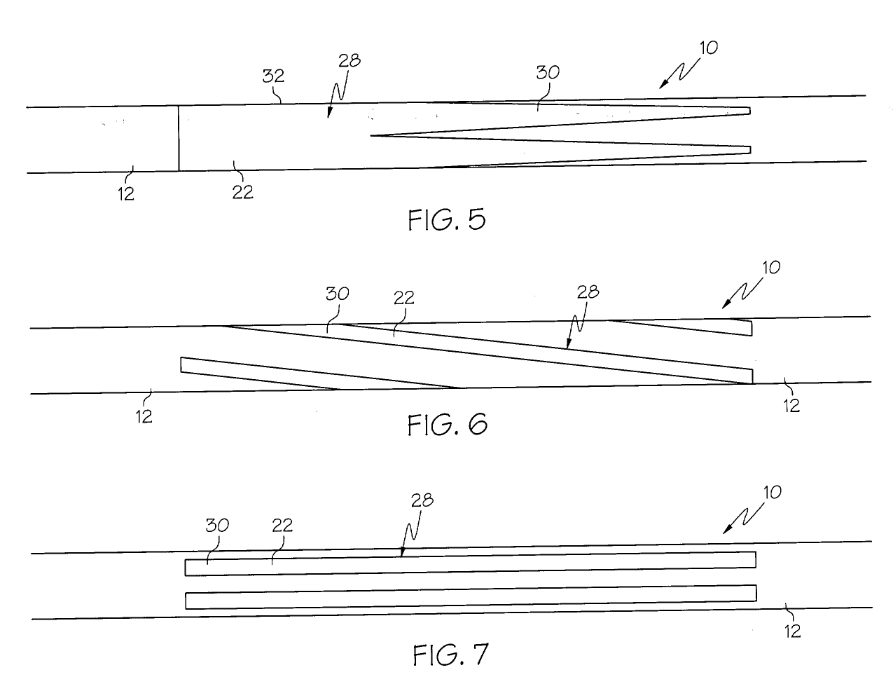

The method shown in FIGS. 1-3 and summarized in FIG. 4, may be utilized to provide a catheter 12 or other tubular member 10 with a variety of surface features and patterns of laser sintered material 28 such as are shown in FIGS. 5-14. The incorporation of laser sintered material 28 into the outer surface 14 of the tubular member 10 allows the physical characteristics of the tubular member to be altered.

For example if the coating 16 comprises a material having a different hardness than that of the material of the tubular member 10, then the portion of the tubular member 10 having the pattern of sintered material 28 will consequently have a reduced or greater hardness than the surrounding material. Such a portion of the tubular member 10 may also be more flexible and/or have improved trackability as a result of the pattern of sintered material. In another example, if the coating 16 contains a metal, particularly a radiopaque metal, which is imbedded into the surface 14 of the tubular member 10 by laser sintering as previously described, the tubular member 10 will be provided with a pattern 22 of sintered material 28 that is radiopaque. By combining a powdered metal, as opposed to a separate metal band or other device, into the tubular material the catheter 12 or balloon 20 may be provided with a radiopacity without significantly compromising flexibility or other material characteristics. By providing the tubular member 10 with sintered material 28 of various composition and/or patterns, a wide variety of physical properties of the tubular member 10 may be greatly modified, enhanced or otherwise altered.

Some examples of patterns 22 of sintered material 28 that a tubular member 28 may be provided with are illustrated in FIGS. 5-14.

In the embodiment shown in FIG. 5 a tubular member 10, such as a catheter shaft 12, includes a pattern 22 of sintered material 28 in the form of a plurality of circumferentially widening stripes 30 that longitudinally extend to form a continuous band 32 of sintered material 28.

In the embodiment shown in FIG. 6, a tubular member 10, such as a catheter shaft 12, includes a pattern 22 of sintered material 28 in the form of one or more longitudinally off set, or helically dispose stripes 30. In the embodiment shown in FIG. 7, a tubular member 10, such as a catheter shaft 12, includes a plurality of longitudinally parallel stripes 30 of sintered material 28.

In the embodiment shown in FIG. 8, a tubular member 10, such as a balloon 20, includes a pattern 22 of sintered material 28 in the form of one or more longitudinally off set, or helically dispose stripes 30.

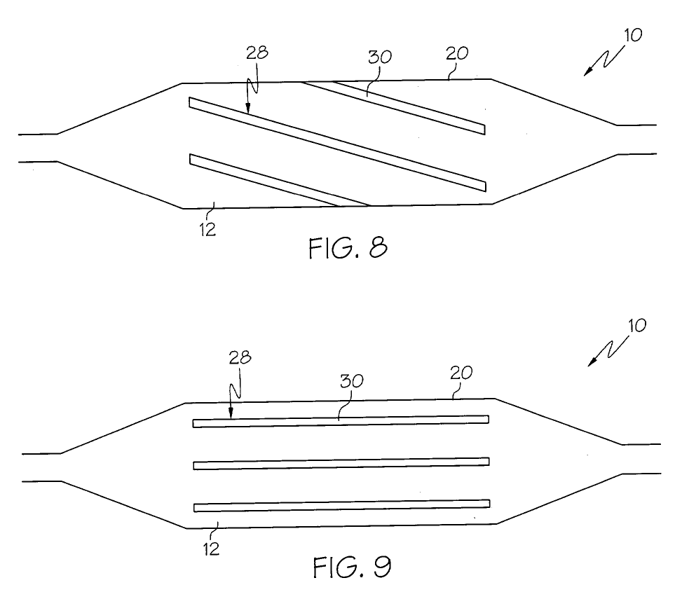

In the embodiment shown in FIG. 9, a tubular member 10, such as a balloon 20, includes a plurality of longitudinally parallel stripes 30 of sintered material 28.

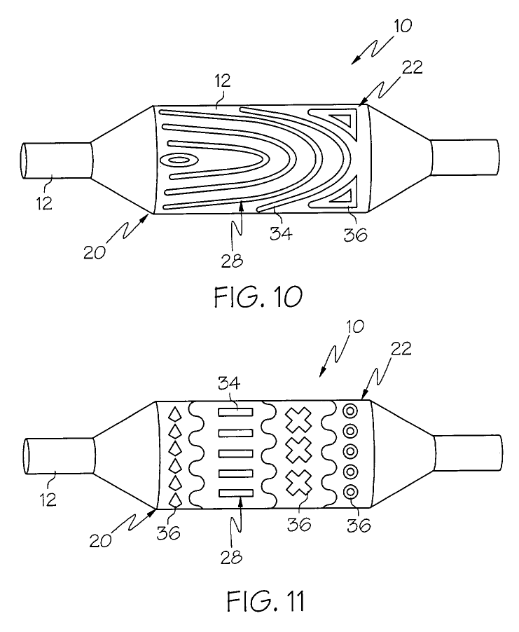

In the embodiments shown in FIGS. 10 and 11, examples of some of the potentially unique patterns 22 of sintered material 28 are shown. As may be seen, a balloon 20 or other tubular member 10 may be provided with a variety of surface features such as elongate ribs 34, and/or the variety of uniquely shaped bumps or protrusions 36. When a pattern 22 of sintered material 28 is provided to the outside surface of a tubular member 10, such as a balloon 20 or catheter 12, the region of the tubular member 10 having the pattern 22 of sintered material 28 may have enhanced stent retaining properties.

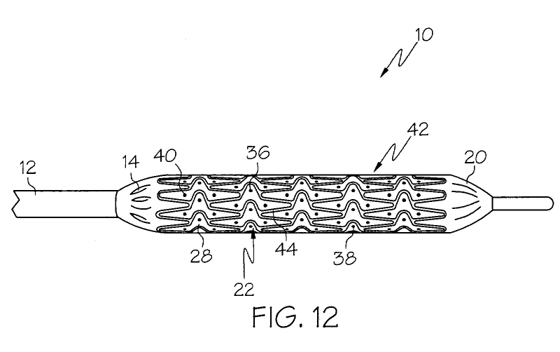

For example, in the embodiment shown in FIG. 12, a catheter 12 is shown having a balloon 20. A stent retaining portion 38 of balloon 20 is equipped with a pattern 22 of raised protrusions 36 of sintered material 28. The placement of protrusions 36 on the balloon surface 14 may correspond with one or more cell openings or spaces 40 of a stent 42 which is disposed about the stent retaining portion 38 of the balloon 20. Prior to delivery of the stent 42, the sintered material 28 may engage the stent members or other stent portions 44 which define the spaces 40 of the stent, and which are adjacent to the protrusions 36.

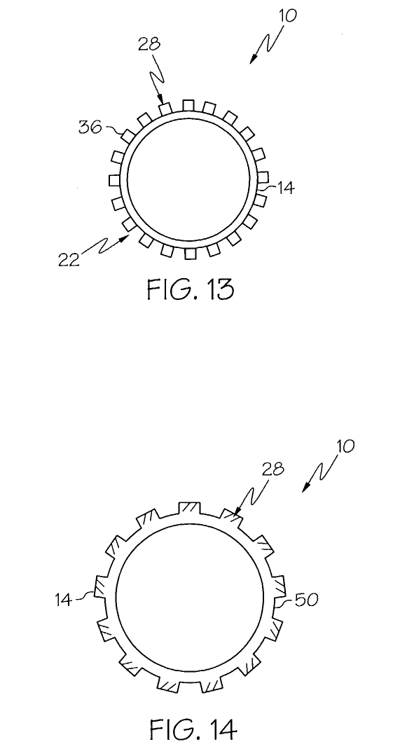

Typically the sintered material 28 will be incorporated into the outer surface 14 of the tubular member 10 to provide a raised protrusions 36 according to a predetermined pattern 22 such as is shown in FIG. 13. If desired however, rather than be used to form protrusions of coating 16 (material 28), laser energy may be utilized to ablate or vaporize existing coatings and/or tubular materials to form depressions or valleys 50 such as are shown in FIG. 14. A tubular member 10 having such valleys 50 may still include sintered material 28 in selected portion of the outer surface 14 depending on the material selected, the particular laser energy applied, and the chosen pattern.

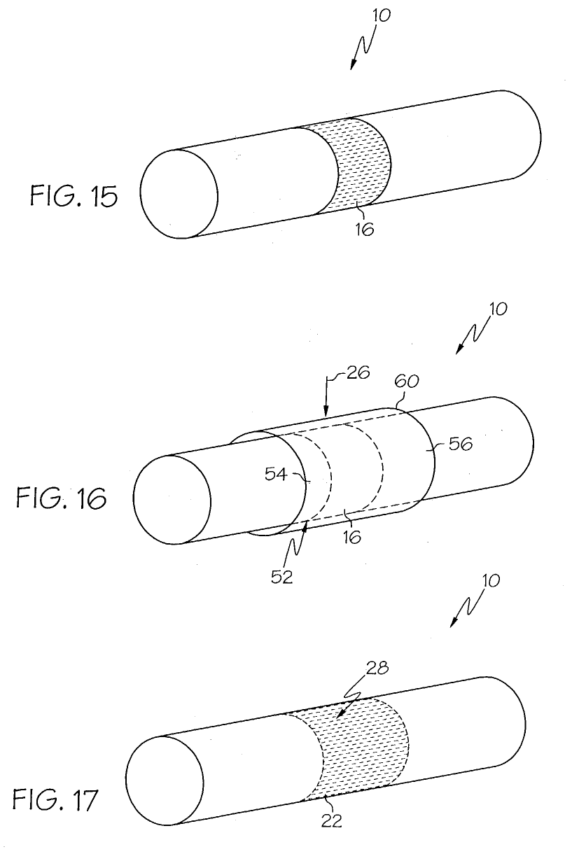

An alternative method of providing a tubular member 10 with sintered material 28 is depicted in FIGS. 15-17. In FIG. 15 the tubular member 10 is shown having a coating 16, such as previously described. A heat shrink 60 is placed about the coated portion 52 of the tubular member 10 as well as at least a proximal portion 54 and distal portion 56 of the tubular member 10 adjacent to the coated portion 52. Laser energy 26, having a wavelength substantially transparent to the heat shrink 60 is then applied to the coating 16 and/or tubular member 10, depending on their respective wavelength absorption properties. The energy 26 is applied until one or more of the coating 16 and tubular member 10 thereunder begin to melt. As the coating 16 and/or tubular member 10 are heated, the heat shrink 60 applies radially inward acting pressure to force the melted matrix of coating 16 and tubular material to be dispersed. When allowed to resolidify the tubular member 10 is provided with a band or other pattern 22 of sintered material 28.

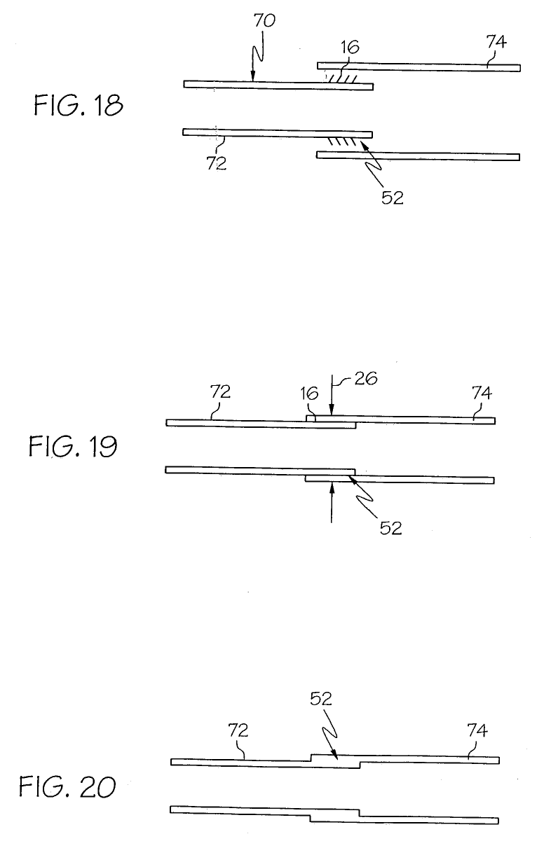

While many embodiments of the invention are directed to providing a tubular member with a pattern of sintered material such as have been described above, other embodiments of the present invention are directed to a novel method for bonding or welding two tubular members together such as depicted in FIGS. 18-20.

In FIG. 18, the outer surface 70 of a first tube 72 is provided with a coating 16 such as has been previously described. A second tube 74 is then disposed about the coated region 52 of the first tube 72. As is shown in FIG. 19, laser energy 26 is then applied to the coated region 52 in order to heat to melting one or more of the coating 16, first tube 72, and second tube 74. Direct heating of one or more of the coating 16, first tube 72, and second tube 74 will cause the remaining elements to also heat and eventually melt as a result of conduction. After all three of the coating 16, first tube 72, and second tube 74 are at least partially melted, the laser energy 26 is removed and the components at and around the coated region 52 are allowed to cool and resolidify, thereby resulting in the substantially seamless joining of the first tube 72 and second tube 74 shown in FIG. 20.

In addition to being directed to the specific combinations of features claimed below, the invention is also directed to embodiments having other combinations of the dependent features claimed below and other combinations of the features described above.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

1. A method of preparing a medical device comprising the steps of:
providing an tubular member having an external surface, the tubular member being constructed of a first material having a melting point;
coating at least a portion of the external surface with a second material having a melting point;
exposing a pattern of at least one of the first material and the second material to laser energy, the laser energy heating the pattern to at least the melting point of at least one of the first material and the second material, the melting point of the first material being different than the melting point of the second material;
removing the laser energy; and
cooling the pattern, thereby sintering the pattern of the second material to the external surface of the tubular member.

2. The method of claim 1 wherein the second material is at least one member of the group consisting of powdered metal, a dispersion of polymer material, and any combination thereof.

3. The method of claim 1 wherein the tubular member consists of a catheter shaft, balloon, sleeve, sheath and any combination thereof.

4. The method of claim 1 wherein the predetermined patter of the second material extends radially outward from the external surface of the tubular member.

5. The method of claim 1 wherein the pattern of the second material is selected from at least one member of the group consisting of: at least on rib, at least one bump, at least one ring, and any combination thereof.

6. The method of claim 1 further comprising the step of:
disposing a template about the tubular member, the template defining at least one opening through which the laser energy accesses the second material, the at least one opening corresponding to the pattern.

7. The method of claim 1 wherein the laser energy is provided by a laser, the laser tracing out a path on the tubular member corresponding to the pattern.

8. The method of claim 1 further comprising the step of:
disposing a heat shrink about the at least a portion of the external surface with a second material.

9. The method of claim 8 wherein the heat shrink is substantially transparent to the laser energy.

10. A medical device comprising:
an tubular member having an external surface, at least a portion of the external surface having at least one raised surface feature laser sintered thereto.

11. The medical device of claim 10 wherein the at least one raised surface feature is constructed from at least one member of the group consisting of powdered metal, a dispersion of polymer material, and any combination thereof.

12. The medical device of claim 10 wherein the tubular member is selected from at least one member of the group consisting of at least a portion of a catheter shaft, at least a portion of a balloon, at least a portion of a sleeve, at least a portion of a sheath and any combination thereof.

13. The medical device of claim 10 wherein the at least one raised surface feature is selected from at least one member of the group consisting of: at least on rib, at least one bump, at least one ring, and any combination thereof.

14. A method of bonding a first tubular member to a second tubular member comprising the steps of:
coating at least a portion of an outside surface of the first tubular member with a sintering material thereby providing a coated region;
disposing at least a portion of the second tubular member about at least a portion of the coated region;
exposing the coated region to laser energy, the laser energy heating at least one of the first tubular member, the second tubular member, and the sintering material to at least the melting point of at least one of the first material, the second material, and the sintering material;
removing the laser energy; and
cooling the coated region.
